(19) United States
(12) Patent Application Publication
(10) Pub. No.: US 2019/0145953 A1
(43) Pub. Date: May 16, 2019

(54) METHODS AND MATERIALS FOR ASSESSING INTESTINAL PERMEABILITY

(71) Applicant: Mayo Foundation for Medical Education and Research, Rochester, MN (US)

(72) Inventors: Michael L. Camilleri, Rochester, MN (US); Madhusudan Grover, Rochester, MN (US); Ravinder J. Singh, Rochester, MN (US)

(73) Assignee: Mayo Foundation for Medical Education and Research, Rochester, MN (US)

(21) Appl. No.: 16/089,844

(22) PCT Filed: Mar. 31, 2017
(86) PCT No.: PCT/US17/25245

§ 371 Date: Sep. 28, 2018

Related U.S. Application Data
(60) Provisional application No. 62/315,910, filed on Mar. 31, 2016.

(51) Int. Cl. G01N 33/493; G01N 33/66; G01N 30/88

(52) U.S. Cl. CPC G01N 33/493; G01N 2030/8813; G01N 30/88; G01N 33/66

(57) ABSTRACT

This document provides methods and materials involved in assessing intestinal permeability of a mammal. For example, methods and materials for administering ¹³C-mannitol to a mammal (e.g., a human) and determining urinary excretion of ¹³C-mannitol as a measure of intestinal permeability of a mammal are provided.

### CROSS-REFERENCE TO RELATED APPLICATIONS

This application claims priority to U.S. application Ser. No. 62/315,910, filed on Mar. 31, 2016. The disclosure of the prior application is considered part of the disclosure of this application, and is incorporated in its entirety into this application.

### BACKGROUND

### 1. Technical Field

This document relates to methods and materials involved in assessing intestinal permeability of a mammal. For example, this document provides methods and materials for administering ¹³C-mannitol to a mammal and determining urinary excretion of ¹³C-mannitol as a measure of intestinal permeability of a mammal.

### 2. Background Information

Measuring intestinal permeability in patients generally involves oral ingestion of molecules that are not metabolized, but rather are excreted in urine where they can be readily measured. Factors influencing excretion of these molecules include the size of the molecule, the charge of the molecule, gut metabolic factors, renal factors, and other factors such as concentration gradient factors, contact time, location of the molecules, the surface area of the small and large intestine, and the potential for degradation of the molecules by digestion or bacterial degradation. Importantly, prior to coming for an intestinal permeability assessment, participants are instructed to not consume any food or drink that contains artificial sweeteners such as Splenda (sucralose), Nutrasweet (aspartame), lactulose, or mannitol for two days prior to and during the visits.

### SUMMARY

This document provides methods and materials involved in assessing intestinal permeability of a mammal. For example, this document provides methods and materials for administering ¹³C-mannitol to a mammal (e.g., a human) and determining urinary excretion of ¹³C-mannitol as a measure of intestinal permeability of a mammal. As described herein, despite being instructed to not consume any food or drink containing mannitol prior to or during an intestinal permeability assessment, measured baseline levels of urinary excretion of mannitol can be high (e.g., greater than 80 μg of mannitol per mL of urine). A high baseline level of mannitol urinary excretion can distort the measured mannitol urinary excretion values determined a subsequent time points after ingestion of a test amount of mannitol. In some cases, up to a third of human patients can exhibit significant levels of mannitol (regular, ¹²C mannitol) detected in the urine at baseline (prior to administration of the test dose). In some cases, the urinary excretion of mannitol in these patients can drop after the administration of a test dose, leading in some cases to erroneous test results.

As also described herein, administration of ¹³C-mannitol and subsequent measurement of urinary excretion of ¹³C-mannitol can be used to assess intestinal permeability. In such cases, the baseline levels of urinary excretion of ¹³C-mannitol can be lower than those observed with mannitol (e.g., ¹²C-mannitol), thereby providing an increased confidence in subsequently measured levels of ¹³C-mannitol.

In general, one aspect of this document features a high performance liquid chromatography method for obtaining mannitol urinary excretion values. The method comprises, or consists essentially of, (a) collecting a first urine sample from a mammal, (b) administering a solution comprising ¹³C-mannitol to the mammal, (c) collecting a second urine sample from the mammal, and (d) using high performance liquid chromatography-mass spectrometry to measure the amount of ¹³C-mannitol present in the first and second urine samples. The mammal can be a human. The solution can comprise lactulose or sucralose. The solution can comprise heavy carbon tagged lactulose or sucralose (e.g., ¹³C-lactulose). The amount of ¹³C-mannitol present in the first urine sample can be less than 1.5 mg. The high performance liquid chromatography can be high performance liquid chromatography-mass spectrometry. The high performance liquid chromatography can be high performance liquid chromatography-tandem mass spectrometry. The second urine sample can comprise all the urine the mammal expelled from zero to about two hours following the administering step. The second urine sample can comprise all the urine the mammal expelled from zero to about eight hours following the administering step. The second urine sample can comprise all the urine the mammal expelled from eight hours to about 24 hours following the administering step. The method can comprise collecting a third urine sample from the mammal and using the high performance liquid chromatography to measure the amount of ¹³C-mannitol present in the third urine sample. The third urine sample can comprise all the urine the mammal expelled for a period of time extending from the time the second urine sample was collected. The period of time can be about four or twelve hours.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A. Mass spectrometric resolution of mannitol isotopes. Multiple reaction monitoring (MRM) mass spectrometry resolves the three mannitol isotopes by mass: ¹²C, the most abundant isotope in nature; ¹³C (1), one carbon atom substituted with one heavy isotope; ¹³C (6), all 6 carbon atoms substituted by the heavy isotope. FIG. 1B. Comparison of ¹³C and ¹²C mannitol as probes to measure intestinal permeability. Excretion values in one representative subject. Urine samples were obtained prior to test sugar ingestion (baseline), and at various time points post-ingestion. Cumulatively excreted concentrations of ¹²C and ¹³C mannitol used as indexes of permeability. ¹³C (6) mannitol was used as an internal standard.

FIG. 2 contains graphs plotting comparisons of ¹²C and ¹³C mannitol at baseline and various intervals after test dose administration.

### DETAILED DESCRIPTION

This document provides methods and materials involved in assessing intestinal permeability of a mammal. For example, this document provides methods and materials for administering ¹³C-mannitol to a mammal (e.g., a human) and determining urinary excretion of ¹³C-mannitol as a measure of intestinal permeability of a mammal.

The methods and materials provided herein can be used to assess intestinal permeability in any appropriate type of mammal. For example, the methods and materials provided herein can be used to assess intestinal permeability in humans, monkey, cows, horses, goats, sheep, dogs, and cats.

In some cases, intestinal permeability can be assessed as follows. A mammal (e.g., a human) can start the assessment in the morning after fasting for about eight hours. In some cases, blood pressure, body temperature, and heart rate can be measured. A baseline urine sample can be collected to establish baseline concentrations of various sugars (e.g., ¹²C-mannitol, ¹³C-mannitol, ¹²C-lactulose, ¹³C-lactulose, ¹²C-sucralose, ¹³C-sucralose, ¹²C-rhamnose, and/or ¹³C-rhamnose). Once the baseline urine sample is collected, the mammal (e.g., human) can ingest a solution containing ¹³C-mannitol alone or a solution containing ¹³C-mannitol in combination with any one of more of ¹²C-mannitol, ¹²C-lactulose, ¹³C-lactulose, ¹²C-sucralose, ¹³C-sucralose, ¹²C-rhamnose, and/or ¹³C-rhamnose. For example, the solution can contain 100 mg of ¹³C-mannitol, 100 mg ¹²C-mannitol, 500 mg ¹²C-lactulose, and 500 mg ¹²C-sucralose in 250 mL of water. In some cases, water can be administered after the solution containing ¹³C-mannitol. For example, about 500 mL of water can be administered about 30 minutes after the solution containing ¹³C-mannitol is ingested to aid in the collection of urine. During the rest of the test period, the mammal (e.g., human) can be allowed to consume water as normally consumed. One or more cumulative urine samples (e.g., one, two, three, four, or more) can be collected over various collection times (e.g., 2 hours, 4, hours, 6 hours, 8 hours, 10 hours, or combinations thereof). For example, cumulative urine samples for 0-2 hours, 2-8 hours, and 8-24 hours from the time the solution was ingested can be collected.

After the urine samples are collected, the level of ¹²C-mannitol, ¹³C-mannitol, ¹²C-lactulose, ¹³C-lactulose, ¹²C-sucralose, ¹³C-sucralose, ¹²C-rhamnose, and/or ¹³C-rhamnose can be determined for each collected urine sample. For example, the levels of ¹³C-mannitol and ¹²C-lactulose can be determined for the baseline urine sample and cumulative urine samples for 0-2 hours, 2-8 hours, and 8-24 hours.

Any appropriate method can be used to determine the level of ¹²C-mannitol, ¹³C-mannitol, ¹²C-lactulose, ¹³C-lactulose, ¹²C-sucralose, ¹³C-sucralose, ¹²C-rhamnose, and/or ¹³C-rhamnose present within a urine sample. For example, high performance liquid chromatography techniques can be used to determine the level of ¹²C-mannitol, ¹³C-mannitol, ¹²C-lactulose, ¹³C-lactulose, ¹²C-sucralose, ¹³C-sucralose, ¹²C-rhamnose, and/or ¹³C-rhamnose present within a urine sample. Examples of high performance liquid chromatography techniques that can be used include, without limitation, high performance liquid chromatography-mass spectrometry and high performance liquid chromatography-tandem mass spectrometry.

In some cases, the amount of ¹³C-mannitol urinary excretion for a 0-2 hour cumulative sample can be used to assess small bowel permeability, and the amount of ¹³C-mannitol urinary excretion for a 8-24 hour cumulative sample can be used to assess colonic permeability. In some cases, ratios (e.g., a ¹³C-mannitol: ¹²C-lactulose or ¹³C-mannitol: ¹³C-lactulose ratio) can be calculated and used to assess intestinal permeability relative to the surface area of the gastrointestinal tract.

The invention will be further described in the following examples, which do not limit the scope of the invention described in the claims.

### EXAMPLES

### Example 1

### Using ¹³C-Mannitol In Vivo to Assess Intestinal Permeability

The following was performed to determine if administration of ¹³C-mannitol can be used to measure intestinal permeability. Ten healthy volunteers (five females) received oral sugars after 8 hours of fasting. Those volunteers using tobacco, NSAIDs, oral corticosteroids, sweeteners, lactulose, mannitol, or medications affecting gastrointestinal transit were excluded. Three saccharides (100 mg of ¹³C-mannitol, 100 mg of ¹²C-mannitol (regular mannitol), and 1000 mg of ¹²C-lactulose (regular lactulose)) dissolved in 250 mL of water were administered. 0-2 hour cumulative mannitol and 8-24 hour cumulative lactulose or mannitol excretion were measured to assess small intestinal and colonic permeability, respectively. After baseline urine collection, collections were pooled for 0-2 hours, 2-8 hours, and 8-24 hours following administration of test sugars. High performance liquid chromatography-tandem mass spectrometry was performed to determine urinary sugar concentrations. Cumulative excretions and lactulose:mannitol (L:M) ratios (ratio of fractional excretion) were calculated. Mann-Whitney test was used for statistical analysis.

¹³C-mannitol exhibited a significantly lower baseline (total mg of ¹³C-mannitol measured from all urine collected just prior to ingestion of the solution containing ¹³C-mannitol) and 8-24 cumulative excretion than the ¹²C-mannitol (Table 1). The 0-2 hour cumulative excretion of ¹³C-mannitol increased 105 times from baseline vs. an increase of 6 times from baseline for ¹²C-mannitol. The calculated 2-8 hour and 8-24 hour L:M ratio was higher using the ¹³C-mannitol as compared to the ¹²C-mannitol. After 100 mg of each form of mannitol was ingested, 24 hour cumulative excretion of ¹³C-mannitol was 31 mg vs. 78 mg for ¹²C-mannitol. Given that an estimated about 75 percent of orally ingested mannitol is metabolized by colonic bacteria, the high cumulative ¹²C-mannitol excretion likely represents inadvertent ingestion prior to or during the test, which impacts the calculated L:M ratios when using ¹²C-mannitol.

TABLE 1
Comparison of urinary excretion parameters for intestinal
permeability using the ¹³C-mannitol and ¹²C-mannitol.

| | ¹³C-mannitol | | ¹²C-mannitol | | p-value |
|---|---|---|---|---|---|
| Cumulative excretion | | | | | |
| (mg): Mean (SEM) | | | | | |
| Baseline (pre-test level) | 0.13 | (0.03) | 2.57 | (0.70) | <0.0001 |
| 0-2 hours | 13.65 | (1.52) | 15.75 | (1.71) | 0.4 |
| 2-8 hours | 12.95 | (2.51) | 25.41 | (6.41) | 0.1 |
| 8-24 hours | 4.38 | (0.76) | 36.47 | (11.52) | <0.0001 |
| Cumulative 24 | 31.21 | (3.40) | 77.63 | (13.06) | 0.0007 |
| hour excretion | | | | | |
| L:M ratio: Mean (SEM) | | | | | |
| 0-2 hours | 0.007 | (0.0006) | 0.006 | (0.0006) | 0.3 |
| 2-8 hours | 0.01 | (0.002) | 0.009 | (0.001) | 0.02 |
| 8-24 hours | 0.01 | (0.002) | 0.002 | (0.001) | 0.002 |

These results demonstrate that use of ¹³C-mannitol significantly eliminates baseline presence of the test sugar and circumvents accidental ingestion of mannitol before or during the testing period. ¹³C-mannitol can serve as a better reagent than ¹²C-mannitol when testing intestinal permeability.

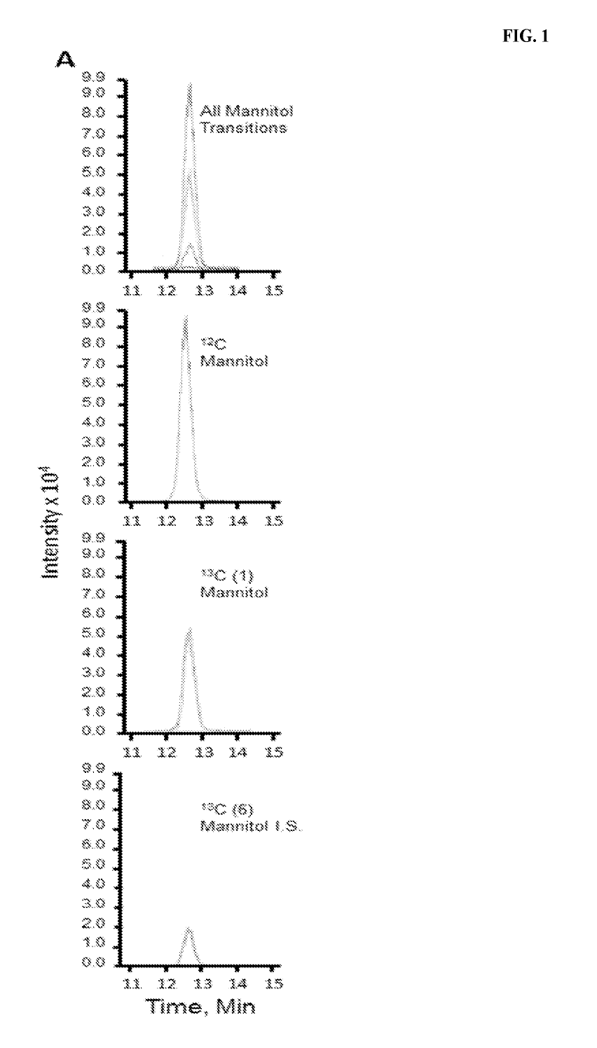

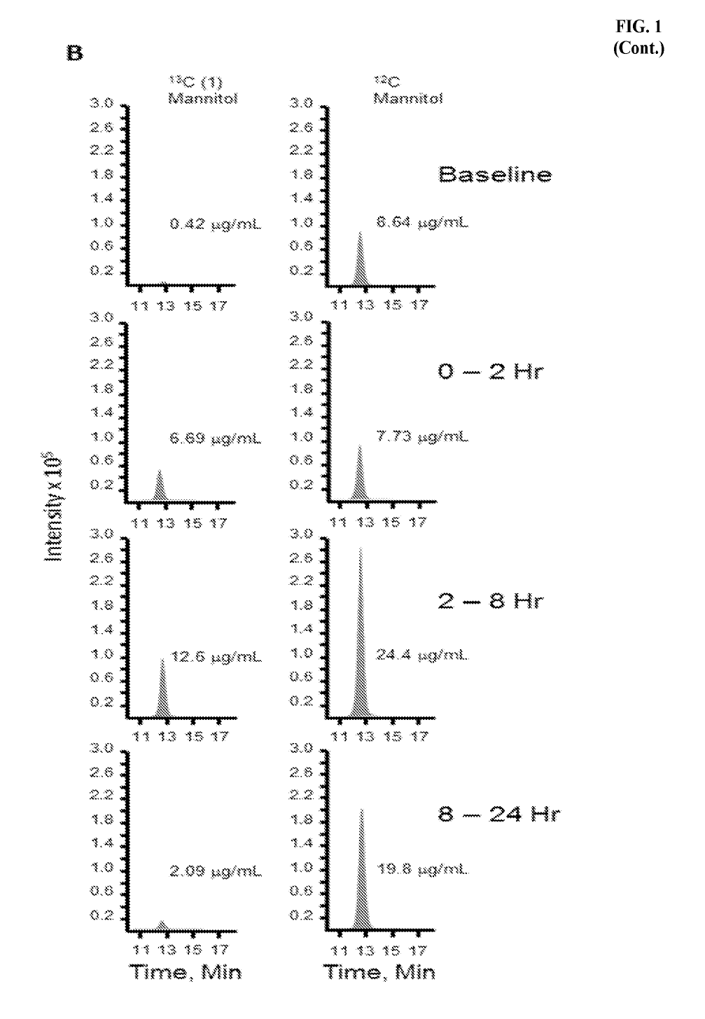

The mannitol isotopes comigrated on liquid chromatography and were separable on mass spectrometry due to the mass shift, as shown from multiple reaction monitoring in FIG. 1A. The limit of detection, the lowest analyte concentration likely to be reliably distinguished from the limit of blank, was 0.018 and 0.051 pg/mL in two lactulose samples tested and 0.021 pg/mL in the mannitol sample tested in multiple replicates. The limit of quantification (LoQ) for lactulose concentration measured reproducibly was 0.063 pg/mL (n=13) with a coefficient of variation (CV) of 17%. The lowest mannitol concentration measured reproducibly was 0.029 pg/mL with a CV of 14%. The LoQ for both analytes was set at 0.3 pg/mL.

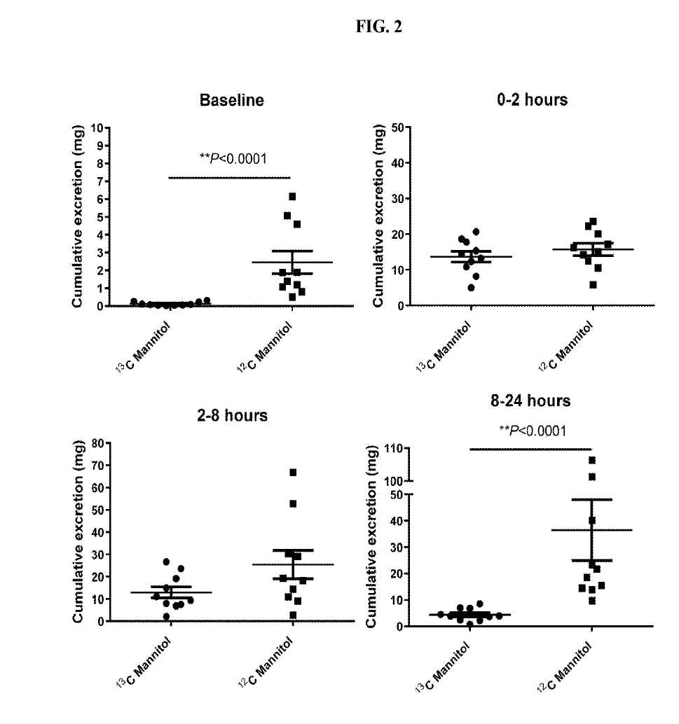

Again, a significantly lower baseline cumulative excretion of ¹³C mannitol than ¹²C mannitol, with a mean 20-fold lower contamination of ¹³C mannitol (Table 1). Three of the ten participants had disproportionately higher baseline excretion of ¹²C mannitol, whereas none of the participants had high ¹³C mannitol excretion at baseline (FIG. 2). The 0-2 hour cumulative excretion of ¹³C mannitol increased 105-fold from baseline in contrast to the sixfold over baseline excretion for ¹²C mannitol, but there was no difference in the actual mass of the two mannitol moieties excreted over this time period. After 100 mg of each form of mannitol was ingested, the overall 24 hour cumulative excretion of the ¹³C mannitol is 31 vs 78 mg for the ¹²C mannitol (Table 1). The excretion of ¹³C mannitol drops between 8-24 hours; however, the ¹²C mannitol excretion continues to peak in that time-frame. This could be possibly due to inadvertent contamination during the study (either from dietary, medications or other sources), baseline sugar competing with absorption or due to the production of ¹²C mannitol by colonic microbiota during the testing. FIG. 1B shows a representative tracing from a urine sample, showing the lower baseline and 8-24 hour excretion of ¹³C mannitol.

### Example 2

### Using ¹³C-Mannitol In Vivo Test to Determine Response to Treatment in Irritable Bowel Syndrome (IBS)

IBS-diarrhea (IBS-D) is associated with low grade inflammation, increased mucosal expression of secretory mechanisms, and disorders of epithelial barrier function.

The following was performed to evaluate safety and effectiveness of oral nutritional therapy with serum-derived bovine immunoglobulin (SBI) on symptoms, epithelial barrier function, and mucosal expression of pivotal genes in small intestinal mucosa (SI) in patients with IBS-D. This open-label pilot study evaluated effects of SBI, 5.0 g twice daily for 8 weeks, in 15 patients with IBS-D (Rome III) on symptoms, tryptophan metabolism (kynurenine to tryptophan ratio), intestinal permeability [measured by two sugars (¹³C-mannitol and lactulose) urine excretion: 0-2 hours (SI) and 2-8 hours (SI and colon)] and distal duodenal mucosal mRNA expression of pivotal genes including tight junction, secretory mechanisms, tissue repair proteins and chemokines. Bowel function and abdominal pain (worst and average) were evaluated using a daily diary including the Bristol Stool Form Scale, and bile acid homeostasis by fasting serum FGF-19 and 7αC4. Statistical analysis was based on ITT principles using paired tests (comparing baseline with results of therapy over 8 weeks or in final 2 weeks) and included imputation for missing data for one patient.

Fifteen Caucasian patients (13 females; 2 males) were enrolled: mean age 40.3+2.3 years and mean BMI 34.3+3.0 kg/m². One patient withdrew (metallic taste in mouth) without completing studies. There was a decrease in average number of stools per day (p<0.001), average stool form (p=0.07), average ease of passage (p=0.035), and incomplete evacuation (p=0.004) after 8 weeks SBI therapy. There were no significant overall effects on pain during 8-week therapy, though the worst pain severity was numerically reduced in the last 2 weeks of treatment (p=0.078). The ¹³C mannitol excretion, as well as lactulose to mannitol excretion ratio (LMR) at baseline in the IBS-D patients did not differ from 8 healthy controls who underwent intestinal permeability test.

There were no significant changes in small bowel or colon permeability after 8 weeks SBI therapy. Although the change in kynurenine to tryptophan ratio from baseline to treatment was not significant (p=0.353), there was a borderline reduction in tryptophan on SBI therapy. Mucosal mRNA expression analyses of 94 candidate genes related to tight junction proteins, immunity and inflammation were not significantly different after SBI therapy. Fold expressions of mRNA of CLDN4 and IL4 genes were 1.18 (p=0.09) and 0.78 (p=0.13), respectively. Serum FGF-19 and 7αC4 were not altered by SBI therapy.

These results demonstrate that SBI therapy for 8 weeks in IBS-D patients is associated with improved bowel function, though the mechanism of benefit is unclear.

### Example 3

### Using ¹³C-Mannitol In Vivo Test to Determine Differences in Permeability Between IBS and Healthy Humans

Duodenal and colonic mucosal barrier function and bacterial translocation in females with constipation predominant IBS were assessed. A subset of patients with diarrhea predominant IBS have increased intestinal permeability. Mucosal barrier function has not been comprehensively studied in constipation predominant IBS (IBS-C).

The following was performed to determine duodenal and colonic barrier function and endotoxin activity in females with IBS-C in comparison to healthy. 16 IBS-C (Rome III) patients and 13 matched healthy volunteers (females) were given oral lactulose, ¹²C mannitol and ¹³C mannitol measured cumulatively over 0-2 and 8-24 hours by HPLC-MS. 10 biopsies were obtained from duodenum and sigmoid colon. Mucosal transepithelial resistance (TER), FITC dextran (4 KDa), and E. coli K12 BioParticle flux were measured in Ussing chambers. Endotoxin activity (blood) was measured towards gram-negative bacterial lipopolysaccharide. Unpaired 2-sided t-tests were used for comparisons.

Mean age was 45.8 years for IBS-C humans vs 44.6 years for healthy humans. The duodenal and colonic TER and flux were not different (Table 2). TER and FITC dextran flux correlated in duodenum (r=−0.58, p=0.001) and colon (r=−0.63, p=0.001). Mean (SEM) endotoxin activity was 0.35 (0.04) for IBS-C humans vs 0.32 (0.03) for healthy humans.

TABLE 2
In vivo permeability, mucosal TER, FITC dextran, and K12 BioParticle flux

| | |
|---|---|
| IBS-C | Healthy Volunteers (HV) |

Duodenum-second part, mean (SEM)

| | | |
|---|---|---|
| 0-2 hr ¹³C mannitol (mg) (IBS-C=16; | 13.04(1.27) | 14.86(1.19) |
| HV = 11) | | |
| 0-2 hr lactulose (mg) (IBS-C = 16; | 1.12(0.19) | 0.99(0.12) |
| HV = 11) | | |
| TER (Ω/cm²) (IBS-C = 15; HV = 13) | 26.90(1.95) | 27.79(2.05) |
| 3 hr FITC-dextran (ng/ml) (IBS-C = 15; | 169.3(37.06) | 181.6(49.25) |
| HV = 13) | | |
| 3 hr E. coli K12 (CFU/ml) (IBS-C = 12; | 18284(6089) | 16752(3939) |
| HV = 9) | | |

Sigmoid colon, mean (SEM)

| | | |
|---|---|---|
| 8-24 hrs ¹³C mannitol (mg) (IBS-C = 16; | 6.04(1.58) | 4.82(0.74) |
| HV = 11) | | |
| 8-24 hr lactulose (mg) (IBS-C = 16; | 1.06(0.55) | 0.72(0.31) |
| HV = 11) | | |
| TER (Ω/cm²) (IBS-C: n = 16; HV: n = 13) | 18.84(1.25) | 15.59(1.35) |
| 3 hrs FITC-dextran (ng/ml) (IBS-C = 12; | 150.9(33.79) | 321.7(153.9) |
| HV = 12) | | |
| 3 hr E. coli K12 (CFU/ml) (IBS-C = 15; | 22773(5601) | 10872(2245) |
| HV = 9) | | |

These results demonstrate that duodenal and colonic barrier function is unaffected in IBS-C. Bacterial translocation and endotoxin levels are also unchanged in IBS-C. Changes in mucosal barrier function likely do not play a role in pathophysiology of IBS-C.

### Example 4

### Using ¹³C-Mannitol In Vivo Test to Determine Changes in Response to NSAID Treatment

The following is performed to show that ¹³C mannitol based in vivo permeability testing demonstrates a response to perturbation using NSAIDs. 24 healthy volunteers (12 females) aged 18-65 years are recruited. Those using NSAIDs, oral corticosteroids, artificial sweeteners (Splenda, aspartame), lactulose, mannitol, or medications affecting gastrointestinal transit are excluded. All 24 participants undergo a baseline urinary excretion assay using the three saccharides (100 mg ¹³C mannitol, 100 mg ¹²C mannitol, and 1000 mg lactulose) dissolved in 250 mL of water. 500 mL of water is administered 30 minutes after the sugars are ingested to aid in the production of urine. Participants are allowed regular amounts of water throughout the study, and no standardized meals are provided. Cumulative urine samples for 0-2 hours are collected while they are in the CRU and stored. Two containers are provided for the cumulative 2-8 hour collection and cumulative 8-24 hour collection. Participants are instructed to return these collections the following day. The collections are subjected to HPLC-MS for quantitative estimation of the sugars.

Subsequently, these participants are allocated to an Indomethacin challenge since Indomethacin has been shown to perturb small intestinal permeability as determined by lactulose/mannitol and ⁵¹Cr EDTA excretion assays. These participants receive 75 mg of Indomethacin orally 8 hour and 30 minutes prior to a second urinary excretion assay using the same three sugars. Finally, these participants undergo a third assay after 4 weeks from the end of the second assay in the absence of any indomethacin or other NSAIDs or perturbations. All three assays are done using the same protocol and urinary collections. Additionally, these participants undergo an upper endoscopy with duodenal biopsies at baseline and after Indomethacin challenge. The ¹³C mannitol-assay based urinary excretion findings are correlated with histology findings on light microscopy and with ex vivo mucosal barrier function/permeability. For this, tw freshly obtained biopsies are mounted in 4 mL Ussing chambers (Physiological Instruments, California, USA) exposing a 0.03 cm² area. Transepithelial resistance is measured using Ag/AgCl electrodes with agar-salt bridges and current delivered with platinum electrodes. Macromolecular flux across biopsies are studied using FITC Dextran (4 kDa, Molecular Probes, New York). At 30-minute intervals for a total of 3 hours, basal compartment fluorescence are analyzed.

End points and statistical analysis: The primary end point for Indomethacin induced small intestinal permeability change and subsequent recovery is the Δ 0-2 hour cumulative excretion of mannitol. The 0-24 cumulative mannitol excretion, 0-2 and 8-24 hour Lactulose:Mannitol excretion ratios are secondary end points. 60-75% of individuals are expected to have an increase in small intestinal permeability following Indomethacin challenge. At least about 15 patients in the Indomethacin treatment group can have a change in the permeability. 2-sided t-tests are used for comparisons of changes in urinary excretions with the ¹²C and ¹³C mannitol. This study provides baseline excretion parameters on 24 healthy subjects and allows calculation of sample sizes for further validation studies in disease states.

### Example 5

### Using ¹³C-Mannitol In Vivo Test to Determine Changes in Response to NSAID Treatment

At each visit, baseline excretion of ¹²C mannitol was higher than the ¹³C mannitol (p<0.0001) with 5 of 73 subjects excreting >10 mg of ¹²C mannitol prior to test dose administration. At visit 1, one subject excreted 62 mg of ¹²C mannitol, which dropped to 28 mg after test dose administration. Additionally, four subjects exhibited no significant increase in excretion in the 0-2 hr interval when compared with baseline.

In comparison, all subjects exhibited nearly undetectable excretion of ¹³C mannitol at baseline, which increased in all cases in the 0-2 hr interval following test-dose administration. Cumulative (0-24h) mean (SEM) recovery of ¹²C mannitol was significantly higher than ¹³C mannitol (p<0.0001) and on average exceeded the administered dose (100 mg) in the case of ¹²C mannitol, likely due to inadvertent ingestion by the participants. Indomethacin challenge resulted in increased 0-2h (p<0.0001), 2-8h (p=0.004), and 8-24h (p=0.004) lactulose excretion compared to baseline (visit 1). This recovered completely at visit 3. Both low and high dose Indomethacin had similar effects. However, ¹²C or ¹³C mannitol excretion did not increase with Indomethacin treatment at either excretion time-point and remained stable at all three visits. It is possible that intestinal microflora can influence the L:M based excretion assay for intestinal permeability. To test this, seven patients underwent a fourth testing following bowel prep with MoviPrep. This resulted in decreased 0-2 hr excretion of ¹³C mannitol (p=0.01) and a trend towards decreased ¹²C mannitol (p=0.06). Additionally, total (0-24 hr) recovery of ¹³C mannitol was decreased (p=0.01), whereas it remained unchanged for ¹²C mannitol (p=0.7) and lactulose (p=0.1). One individual excreted significantly more, and another individual excreted significantly less ¹²C mannitol following bowel prep. However, all individuals consistently excreted a significantly less amount of ¹³C mannitol after the bowel prep. This could reflect increased transit after the bowel prep resulting in less contact time between the sugar and the absorptive surface area.

These results demonstrate that ¹³C mannitol is not present in baseline urine samples, whereas a subset of participants excrete significant amount of ¹²C mannitol at baseline, which can confound the testing. These results also demonstrate that the 115 mg 24h urinary recovery of ¹²C mannitol (following 100 mg administration) reflects likely contamination during the testing period. However, ¹³C mannitol recovered (30 mg), which is consistent with the expected turnover by the microbiome (about 75% consumption). In addition, these results demonstrate that Indomethacin (low or high dose) increases lactulose excretion, but does not affect mannitol excretion. Further, bowel prep had a consistent effect of decreasing ¹³C mannitol excretion, however, ¹²C mannitol excretion can increase, decrease, or remain stable. In addition, these results demonstrate that intestinal permeability measurements using lactulose mannitol excretion is temporally stable in healthy volunteers and ¹³C based assay is unlikely to be confounded by inadvertent dietary consumption or microbial factors.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A high performance liquid chromatography method for obtaining mannitol urinary excretion values, wherein said method comprises:
(a) collecting a first urine sample from a mammal,
(b) administering a solution comprising ¹³C-mannitol to said mammal,
(c) collecting a second urine sample from said mammal, and
(d) using high performance liquid chromatography to measure the amount of ¹³C-mannitol present in said first and second urine samples.

2. The method of claim 1, wherein said mammal is a human.

3. The method of claim 1, wherein said solution comprises lactulose.

4. The method of claim 1, wherein said solution comprises ¹³C-lactulose.

5. The method of claim 1, wherein said amount of ¹³C-mannitol present in said first urine sample is less than 1.5 mg.

6. The method of claim 1, wherein said high performance liquid chromatography is high performance liquid chromatography-mass spectrometry.

7. The method of claim 1, wherein said high performance liquid chromatography is high performance liquid chromatography-tandem mass spectrometry.

8. The method of claim 1, wherein said second urine sample comprises all the urine said mammal expelled from zero to about two hours following said administering step.

9. The method of claim 1, wherein said second urine sample comprises all the urine said mammal expelled from zero to about eight hours following said administering step.

10. The method of claim 1, wherein said second urine sample comprises all the urine said mammal expelled from eight hours to about 24 hours following said administering step.

11. The method of claim 1, wherein said method comprises collecting a third urine sample from said mammal and using said high performance liquid chromatography to measure the amount of ¹³C-mannitol present in said third urine sample.

12. The method of claim 11, wherein said third urine sample comprises all the urine said mammal expelled for a period of time extending from the time said second urine sample was collected.

13. The method of claim 12, wherein said period of time is about four hours.
